(19) United States
(12) Patent Application Publication
(10) Pub. No.: US 2026/0020818 A1
(43) Pub. Date: Jan. 22, 2026

(54) TRAUMA RESILIENCY PROTOCOL (TRP) AND EMOTIONS MANAGEMENT PROCESS FOR ADDRESSING POSTTRAUMATIC STRESS

(71) Applicant: Daniel JARVIS, Winter Haven, FL (US)

(72) Inventor: Daniel JARVIS, Winter Haven, FL (US)

(21) Appl. No.: 19/270,951

(22) Filed: Jul. 16, 2025

Related U.S. Application Data
(60) Provisional application No. 63/672,048, filed on Jul. 16, 2024.

(51) Int. Cl. A61B 5/00; A61B 5/16

(52) U.S. Cl. CPC A61B 5/4848; A61B 5/165

(57) ABSTRACT

A therapeutic protocol and method for addressing posttraumatic stress and other negative emotional states through visualization techniques, dissociation, neuro-linguistic programming (NLP), and reimprinting of emotions. The protocol, known as the Trauma Resiliency Protocol (TRP) and Emotions Management Process (EMP), mitigates the psychological impacts of traumatic events. The protocol applies NLP principles, uses both secular and faith-based scripts, and incorporates specific grounding exercises to ensure emotional stability. The protocol emphasizes non-clinical interventions and peer coaching, making the protocol accessible for use by mental health professionals, peer coaches, and individuals suffering from PTSD, C-PTSD, and other related conditions. The protocol is applicable in both clinical and non-clinical settings.

### CROSS REFERENCE TO RELATED APPLICATIONS

This application claims benefit to U.S. Provisional Patent Application 63/672,048 filed Jul. 16, 2024, the disclosure of which is incorporated herein in its entirety by reference.

### FIELD OF TECHNOLOGY

The present disclosure relates to therapeutic protocols and methods for addressing post-traumatic stress and other negative emotional states. Specifically, the disclosure related to using visualization techniques, dissociation, neuro-linguistic programming (NLP), and reimprinting of emotions to mitigate the psychological impacts of traumatic events.

### BACKGROUND

Posttraumatic stress disorder (PTSD) has been a recognized diagnosis since 1980. The condition is typically assessed using tools such as the PSSI-5, which determines qualification based on a scoring system. There is an ongoing debate about renaming PTSD to Posttraumatic Stress Injury (PTSI) to reduce stigma and better reflect the condition's nature. High suicide rates among U.S. armed forces, veterans, and first responders highlight the need for effective treatments. PTSD is observable through brain mapping techniques like quantitative electroencephalography (QEEG), indicating biological alterations in the nervous system, particularly the sympathetic system.

Complex PTSD (C-PTSD) is not yet an official diagnosis but includes symptoms such as difficulties in relationships, emotional regulation, and altered attention. These symptoms often begin in early childhood and persist through multiple traumas. Traditional therapies for PTSD and C-PTSD have limitations, necessitating new approaches to treatment.

### SUMMARY

The embodiments provide a unique approach to addressing and mitigating the emotional and psychological impacts of traumatic events through a structured protocol. This protocol, known as the tactical resiliency protocol (TRP) and Emotions Management Process (EMP), utilizes a combination of visualization techniques, dissociation, and reimprinting of emotions to effectively reduce or eliminate the distress associated with traumatic memories. The disclosure is particularly innovative in its application of NLP principles, the use of both secular and faith-based scripts, and the incorporation of specific grounding exercises to ensure the emotional stability of the client throughout the process. Additionally, the disclosure's emphasis on non-clinical interventions and peer coaching makes it accessible and practical for a wide range of users, including military personnel, first responders, and individuals suffering from PTSD and other negative emotional states.

According to one aspect of the present disclosure, a TRP and EMP for addressing posttraumatic stress utilizes a combination of visualization techniques, dissociation, and reimprinting of emotions to reduce or eliminate distress associated with traumatic memories.

According to another aspect, the protocol applies NLP principles, uses both secular and faith-based scripts, and incorporates specific grounding exercises to ensure emotional stability throughout the process.

According to yet another aspect, the protocol emphasizes non-clinical interventions and peer coaching, making it accessible and practical for a wide range of users, including military personnel, first responders, and individuals suffering from PTSD and other negative emotional states.

The disclosure is applicable in both clinical and non-clinical settings, making it accessible for use by mental health professionals, peer coaches, and individuals suffering from PTSD, C-PTSD, and other related conditions.

The present disclosure relates to therapeutic protocols and methods for addressing post-traumatic stress and other negative emotional states. Specifically, it involves the use of visualization techniques, dissociation, NLP, and reimprinting of emotions to mitigate the psychological impacts of traumatic events. The disclosure is applicable in both clinical and non-clinical settings, making it accessible for use by mental health professionals, peer coaches, and individuals suffering from PTSD, C-PTSD, and other related conditions.

### BRIEF DESCRIPTION OF THE DRAWINGS

Illustrative embodiments may take form in various components and arrangements of components. Illustrative embodiments are shown in the accompanying drawings, throughout which like reference numerals may indicate corresponding or similar parts in the various drawings. The drawings are only for purposes of illustrating the embodiments and are not to be construed as limiting the disclosure. Given the following enabling description of the drawings, the novel aspects of the present disclosure should become evident to a person of ordinary skill in the relevant art(s).

FIG. 1 illustrates an exemplary peer rescue protocol in accordance with embodiments of the present.

FIG. 2 illustrates a flowchart an exemplary method of practicing peer rescue protocol depicted in FIG. 1 in accordance with the embodiments.

FIG. 3 illustrates exemplary applications of tactical resiliency training (TRT) in accordance with embodiments.

FIG. 4 illustrates a scan of a brain having an activated amygdala and an active neocortex in accordance with the embodiments.

FIG. 5 illustrates an individual in an environment capable of creating the need for one or more embodiments of the present disclosure.

FIG. 6 illustrates the deployment of first responders in environments that may necessitate the application of one or more embodiments of the present disclosure.

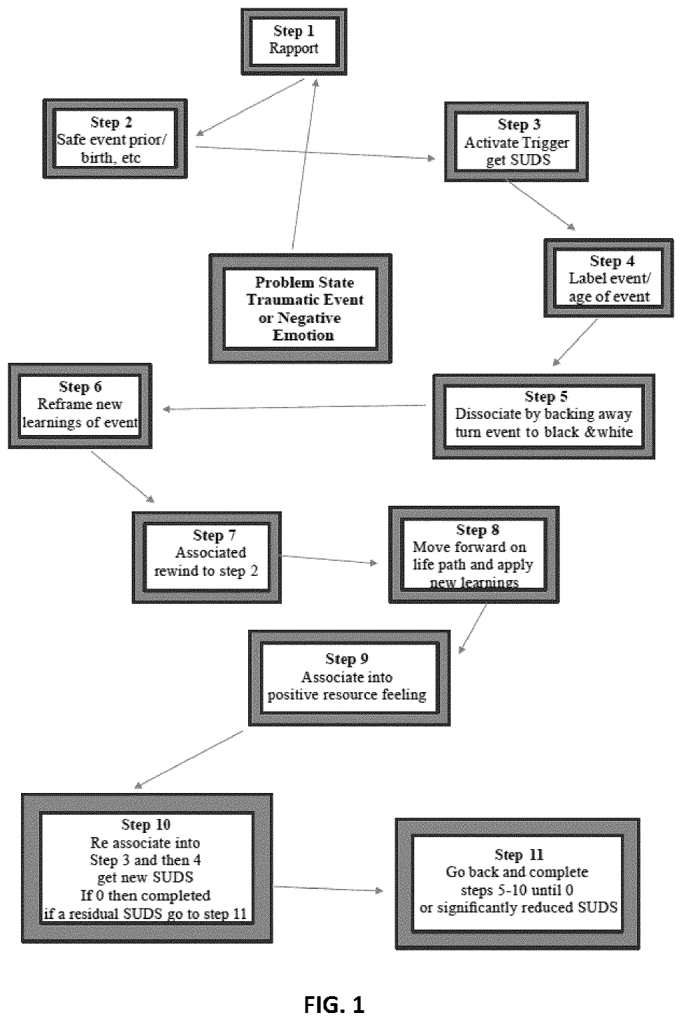

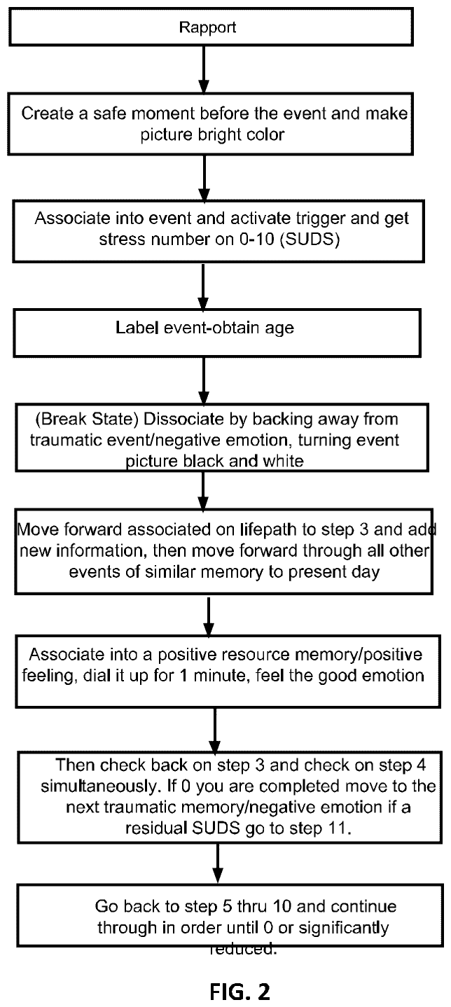

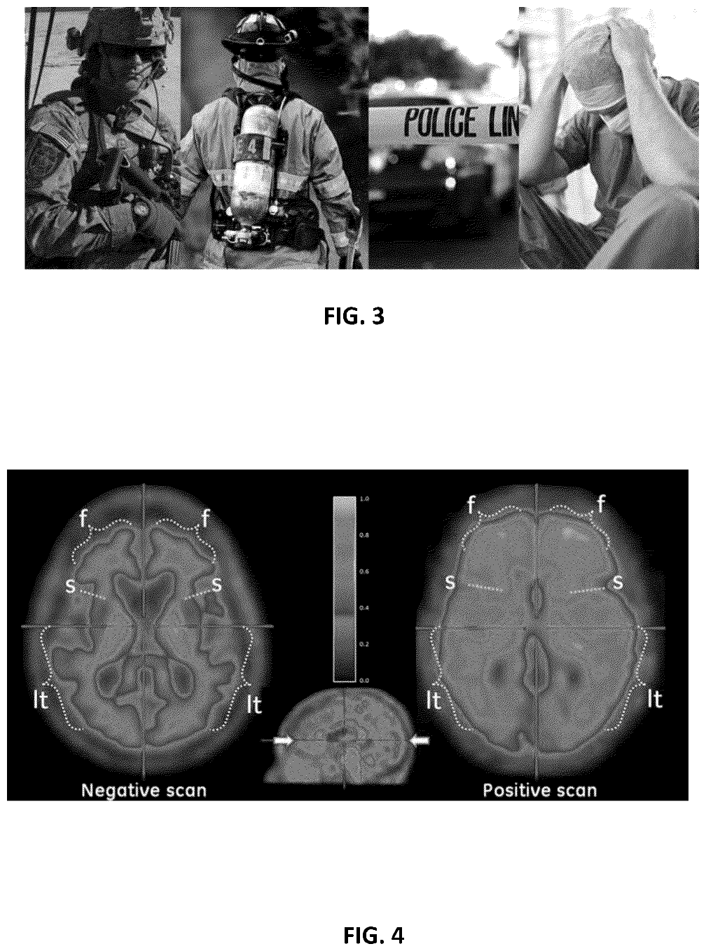

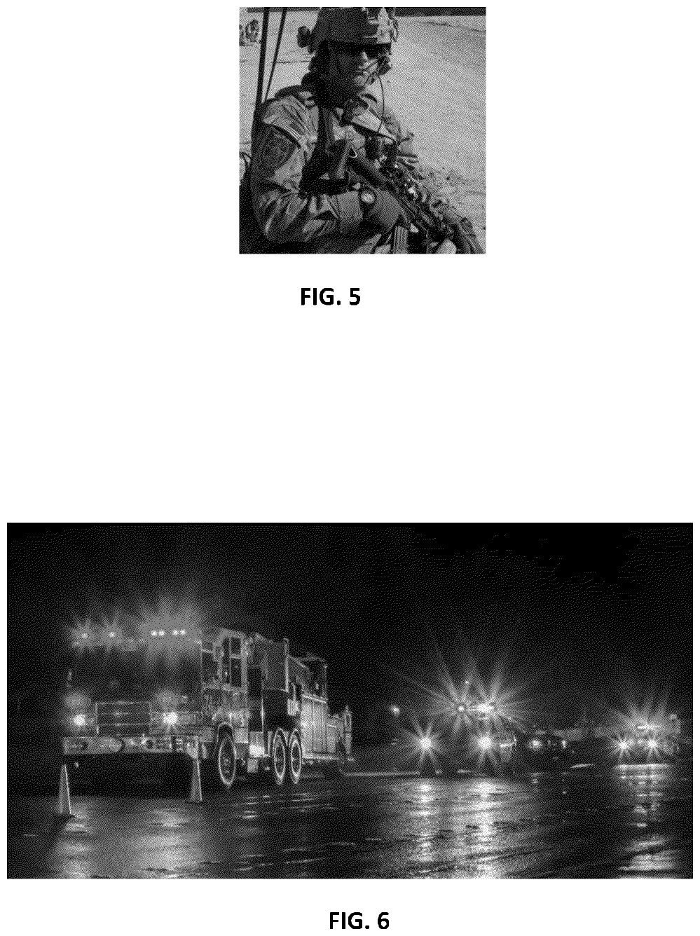

### DETAILED DESCRIPTION

While the illustrative embodiments are described herein for particular applications, it should be understood that the present disclosure is not limited thereto. Those skilled in the art and with access to the teachings provided herein will recognize additional applications, modifications, and embodiments within the scope thereof and additional fields in which the present disclosure would be of significant utility.
TRP—Secular Script
TRP—Jesus Protocol Script
EMP-Secular Script
EMP-Faith Based Emotional Re-Framing
Positive Trauma Secular Script
Positive Trauma Faith-Based
Practice Visual Exercise

### PTSD

PTSD has been an accepted diagnosis since 1980. The PSSI-5 (E. B. Foa et al., 2015), the posttraumatic scale interview version 5 assessment tool, is used to determine the qualification for PTSD. If a client answers the questions and scales above 20, the person qualifies for a diagnosis. We believe it is time to adopt a new name: Posttraumatic Stress Injury. PTSI is a more accurate, hopeful, and honorable name.

Dr. Frank Ochberg, MD, was one of the original editors of the Diagnostic and Statistical Manual (DSM), which named PTSD an illness. Dr. Ochberg has been trying to change the diagnosis name to Post Traumatic Stress Injury (PTSI) because of the stigma immediately attached.

With suicide rates in the U.S. armed forces, veterans, and first responders at record highs, the debate is raging about whether changing to PTSI would help reduce stigma. PTSD is observable in Brain Mapping with QEEG; as Trauma, it may respond to an approach that reverses or treats biological alteration of the nervous system. The division of the nervous system, which is likely to play a dominant role in the development and maintenance of PTSD, is the sympathetic system (Global Post Traumatic Stress Injury Foundation, 2015).

According to the DSM-V, Complex PTSD is not yet an official diagnosis; efforts are underway to include this broadened definition. Complex PTSD symptoms include difficulties in relationships and belief systems, difficulty regulating Emotions, altered attention (dissociation and amnesia) consciousness, somatic distress (headaches, pain, nausea), hyperarousal, being on edge, avoidance and avoidance of reminders, flashbacks, nightmares, and intrusive thoughts. C-PTSD typically begins in early childhood, persisting forward with multiple traumas accumulated.

### Subconscious Mind

The subconscious mind processes information and memories outside our conscious awareness. It plays a crucial role in shaping our thoughts, emotions, and behaviors. Sigmund Freud developed one of the earliest theories of the subconscious mind in the late 19th and early 20th centuries. Freud believed that the subconscious mind contains all our repressed thoughts, emotions, and experiences, and these repressed thoughts and emotions can influence our behavior and mental states. He proposed the idea of the “unconscious” mind as a reservoir of forgotten memories, experiences, thoughts, and feelings that are outside conscious awareness but still influence our behavior and emotions. Recent research in neuroscience and psychology has provided more insight into the functioning of the subconscious mind. Studies using brain imaging techniques, such as functional magnetic resonance imaging (fMRI), have shown that the subconscious mind actively processes information and memories even when we are unaware of it.

It has been found that the subconscious mind plays an important role in regulating our emotions, controlling our behaviors, and maintaining our physical health. For example, research has shown that our subconscious mind regulates our heart rate, blood pressure, and other physiological processes.

Additionally, the subconscious mind plays a crucial role in our decision-making process. Research has shown that the subconscious mind can make decisions much faster than the conscious mind and that past experiences, emotions, and memories can influence these decisions.

The subconscious mind can also be influenced by various forms of therapy, such as hypnotherapy and cognitive-behavioral therapy. These therapies can help individuals access and process the repressed thoughts and emotions stored in the subconscious mind and can help to change negative patterns of thinking and behavior.

In conclusion, the subconscious mind is integral to our mental and emotional functioning. It plays a crucial role in shaping our thoughts, emotions, and behaviors and regulates our physiological processes and decision-making. Further research is needed to fully understand the workings of the subconscious mind, but it is an essential part of our mental and emotional well-being.

### Creative Subconscious Mind

The creative Subconscious refers to the part of the mind responsible for generating new ideas, insights, and solutions. It is the source of our intuition, imagination, and creativity. The Subconscious can process vast amounts of information and make connections that the conscious mind may not be aware of. It is also responsible for our emotional responses and memories.

Accessing the power of the creative Subconscious can be done through various methods such as meditation, hypnosis, guided imagery, and other relaxation techniques. These methods can help an individual reach a state of mind that allows the Subconscious to surface.

One of the most popular methods is brainstorming, a technique used to generate new ideas and solutions by allowing the mind to associate freely. This technique can be done individually or in a group setting, and it's based on the idea that the subconscious mind can come up with new ideas and insights when the limitations of the conscious mind do not constrain it.

Another way to access the power of the creative Subconscious is through metaphor. Metaphors are used to describe one thing in terms of another and can help individuals understand complex ideas in a new and unique way. This way can be helpful for individuals who are looking to solve problems or come up with new ideas.

The power of the creative Subconscious can also be accessed through mindfulness, which is the ability to be present in the moment and pay attention to thoughts and feelings without judgment. Mindfulness allows individuals to be more aware of their subconscious thoughts, feelings, and emotions and access the information stored in the Subconscious.

### Conscious Mind

The conscious mind is responsible for our awareness of the present moment and our ability to think, reason, and make decisions. It is the part of the mind we know and can actively control.

The concept of the conscious mind can be traced back to the ancient Greek philosopher René Descartes, who, in the 17th century, proposed that the mind and the body are separate entities and that the mind is responsible for our ability to think and reason.

Recent research in neuroscience and psychology has provided more insight into the functioning of the conscious mind. Studies using brain imaging techniques, such as fMRI, have shown that the conscious mind is associated with specific areas of the brain, particularly the prefrontal cortex.

The conscious mind is responsible for various mental processes, including attention, perception, memory, and decision-making. It enables us to focus on specific tasks, perceive and interpret our surroundings, remember past experiences, and make decisions based on our goals and values.

The conscious mind is also responsible for our ability to reflect on our thoughts, emotions, and behaviors and make intentional changes, which is the foundation of many forms of therapy, such as cognitive-behavioral therapy, which aims to help individuals understand and change their thought patterns and behaviors.

However, the conscious mind is limited in its capacity and can be easily overwhelmed by too much information or too many tasks, which is where the subconscious mind comes in. It can process information and memories outside our conscious awareness, allowing the conscious mind to focus on more pressing tasks.

In conclusion, the conscious mind is essential to mental and emotional functioning. It is responsible for our awareness of the present moment and ability to think, reason, and make decisions. It allows us to reflect on our thoughts, emotions, and behaviors and make intentional changes. Further research is needed to fully understand the workings of the conscious mind, but it plays a vital role in our mental and emotional well-being.

### Reticular Activating System

The reticular activating system (RAS) is a network of neurons in the brainstem that plays an important role in regulating arousal and attention. It is responsible for controlling the activity level in the brain, helping filter out unnecessary information, and allowing us to focus on important tasks.

The RAS is composed of several nuclei that are responsible for different functions. The locus coeruleus, for example, is responsible for releasing norepinephrine, a neurotransmitter involved in attention and arousal. On the other hand, the dorsal raphe nucleus is responsible for the release of serotonin, which plays a role in mood regulation.

The RAS regulates the sleep-wake cycle, allowing us to transition between different states of consciousness. It also regulates the autonomic nervous system, helping control heart rate, blood pressure, and other vital functions.

The RAS is also involved in filtering sensory information, allowing us to focus on important stimuli while ignoring irrelevant information. It also helps to regulate attention and memory, allowing us to store and retrieve important information.

RAS is also associated with certain disorders and diseases. For example, dysfunction in the RAS can contribute to insomnia, depression, and attention deficit hyperactivity disorder (ADHD).

Exercise: prior to bedtime write down the 3 best things that happened that day and why you think they happened. Then read it in the morning upon waking. It will shift the RAS filter after a couple of weeks. They will start seeing the good instead of the negative things.

### TRP Overview

The TRP is a brain-based method to develop subconscious resiliency skills in the brain, allowing for a neurological disconnect from traumatic emotions and memories. This process removes the emotions associated with traumatic memories, typically fear, terror, and helplessness. The client will remember the event; however, associated emotions will re-consolidate where the feelings and memory belong in the brain. They will be just normal memory stored in the long term.

The coach will guide the client through systematic visualization exercises during the TRP session. The protocol will allow the coach to use visual formats to sever the emotions associated with a traumatic memory. The coach is not involved in any diagnosis or counseling. When the trauma releases and the individual no longer exhibits symptoms of PTSD, they should seek treatment from a qualified mental health counselor or psychologist for an evaluation to determine the need for additional therapeutic services. Under no circumstances do they stop taking prescribed medications without consulting their physician.

The separation of emotion from the traumatic event(s) will occur after using a series of visual imaging exercises. The coach will learn the skills necessary to gauge the individual's reactions. These are changes in breathing, elevated blood pressure, skin color changes, heightened voice, change in tempo, rapid eye movement, or rapid movement of an extremity to know when the visual formatting starts. The coach will walk the individual through the scripted exercises until they are proficient and do not need the script.

Once the coach recognizes the reactions, they will learn different techniques to keep the client calm and comfortable. The client works through the TRP, developing the skills necessary to develop resiliency on the subconscious level, allowing a rapid bounce back to healthy homeostasis.

The coach will learn each reaction type to offer the proper feedback to assist the client in remaining calm enough to complete the protocol. The coach will lead the client out of the uncomfortable emotional state and back to a healthy, grounded state, which is critical to maintaining an emotionally detached state. The individual should not relive the traumatic event fully associated with the protocol. It is especially important to ensure the correct outcome of the TRP. When the individual is comfortable and re-grounded, the coach will reinsert them into the dissociated scenario and continue the process until they are completely comfortable.

### EMP Overview

A secondary training structure allows the skills necessary to release negative emotions connected to specific events through the Emotion Management Process (EMP). The individual being coached/counseled is not required to speak about the events; however, they can reframe each emotion with current levels of understanding at the root of Negative Emotion. This process often renders sympathetic emotional reactions to lifelong negative emotions like Anger, sadness, fear, shame, hurt, abandonment, and anxiety, which may have been present since early childhood, by releasing them at the onset of the emotions using framing exercises.

To become certified in training, the coach must meet two people and successfully coach through the TRP and EMP outside the instructional classroom training. When the training is complete in the classroom, the coach will receive 24 hours of credit cutting-edge

Tactical Resiliency Training consisting of TRP and EMP. After the training is completed and the coach has executed TRP/EMP with two individuals, the coach must provide feedback to the coach's training certifier. They need to use an appropriate scoring metric (PSSI-5, PCL-5, PCL-C, PHQ-9, GAD-7), SUDS evaluation pre and post, and SUDS successfully reduced 90percent or more in TRP/EMP. After showing competence with the delivery of TRP and EMP, the coach will have met certification requirements.

The Significance of the number 22. 22 Veterans, military members, or members of the National Guard or Military Reserves will end their lives today, tomorrow, and in the coming days unless something changes. Our mission is to reduce the number of suicides in the active military, military veteran, and first responder communities through education, connection, and better trauma treatment.

In September 2018, Dan Jarvis connected with a research nonprofit in Albuquerque, NM. Dan learned of their new treatment for Trauma, which is an absolute breakthrough to current counseling methods. This new treatment was designed for the clinical world and hard science to support their claims of 92 percent success at ending PTSD. We have funded more than 20 licensed counselors in this process. Dan continued searching for better ways to work with PTSD and negative emotions and developed the Tactical Resiliency Process; significant changes have occurred since its inception.

The EMP modeled an NLP with a hypnotic language pattern based on gestalt therapy first developed by Fritz Pearls. Our first testing of the model study came in at 100 percent success at eliminating PTSD symptoms, which seemed way too good to be true and is a Tactical Resiliency USA study, not independent. We then conducted a “Validating the Model” study with another one-hundred-person study that replicated the first study's success as we refined the process. A third study with 66 children also replicated the 100 percent success of eliminating the symptoms of PTSD. The child study's remarkable nature is that 78 percent of the 66 children only required one (1) session to resolve trauma symptoms. There is more to follow.

EMP to work with all other negative emotions that do not rise to the level of PTSD or may not be traumatic but problematic; this is a work in progress. The EMP is rooted in Neuro-Linguistic Programming, with the Milton Model Hypnotic language patterns with presuppositional language patterns drawn from the META Model. We are witnessing the same level of remarkable success when resolving negative emotions outside the trauma spectrum using EMP.

In creating TRP, we modeled the early 1980 s process called Visual/Kinesthetic Dissociation (VK/D). Dr. Richard Bandler and Dr. John Grinder replicated the VK/D. VK/D, these processes were founded as early as the 1950 s; however, Neuro-Linguistic Programming (NLP) did not have a track record of acceptance in the mainstream Psychology World until recently. Research has been conducted at very high levels, and new research is underway.

We recognize the necessity to conduct research, our own and independent study outside of Tactical Resiliency USA, to allow more mainstream acceptance of our processes. We recognize the licensed world of mental health as a huge ally in ending suicide in the ranks of veterans, military, and first responders. The military terminology of a Licensed Professional Counselor/Psychologist is the force multiplier.

Our goal in the resiliency training is to use zero details about traumatic or extreme negative emotions to avoid vicarious Trauma with the coach. The coaches are not permitted to dig into the details. However, licensed professionals may need to dig deeper, especially with the reframing part of EMP, to allow for the necessary learnings to help the client release the negative emotion. Coaches can achieve the same results; coaches may have to work harder to get the learnings necessary to facilitate a release. We are helping the client develop resiliency skills to bounce back from trauma or extremely negative emotions, teaching their brain a new skill.

### The Tactical Resiliency USA Founder's Story “We are our Brothers and Sisters' Healers.”

Retired Army SFC Dan Jarvis was deployed to a combat zone in Afghanistan in 2011. Shortly after his arrival, he stepped on a pressure plate and detonated an Improvised Explosive Device (IED) five feet from his position on the patrol. The IED inflicted a traumatic brain injury. He insisted on leading his troops on the battlefield instead of taking a break; however, the Battalion surgeon ordered him out for a week before rejoining his Soldiers. Three weeks after the IED attack and after no sleep, he led his Platoon as lead Stryker in a convoy operation to escort an explosive team to defuse a bomb found by another Platoon. His role in the lead vehicle as the truck commander (TC) was to detect IEDs on the route to ensure safe passage for the other vehicles in the convoy. An undetected IED exploded under the fourth vehicle in the convoy, taking the life of SPC Doug Cordo. Dan felt responsible for losing Doug.

Dan received a Red Cross message informing him that his mother was deathly ill near the end of his deployment. He immediately left Afghanistan and headed to the United States, hoping to see his mom before she took her last breath. Unfortunately, he did not make it in time to say goodbye before she died, which weighed heavily on him. After her funeral, Dan returned to his assigned duty station in Fairbanks, Alaska, at Ft. Wainwright. Burdened under such guilt and still unable to sleep, Dan self-medicated with alcohol, binge drinking each night until he fell asleep; this became his norm.

On Mar. 2, 2013, Dan contemplated suicide. He was eyeing a rifle in the corner and convincing himself that it would be easy to end the suffering. Dan heard the pitter-patter of little feet on the floor of the apartment above him, and he realized firing the rifle would potentially endanger those kids or their parents. He passed out from the alcohol, waking up the next morning to a phone call informing him that one of the soldiers from his former Platoon,

SPC Corey Smathers, had shot and killed himself the night prior. Questioning how they missed Smathers's struggles, Dan realized that he was masking the signs from those he knew and loved. Dan and the other men struggled as they grieved their brother and Soldier. Dan resolved that he would not take his own life because of watching the men struggle to lose Corey and worrying that he may “green light” one of them to do the same. However, the emotional scars remained, as did the binge drinking each night.

Due to multiple combat injuries, Dan medically retired from the Army on Sep. 11, 2014, and returned home to Winter Haven, Florida. He quickly busied himself by reentering the law enforcement profession. All the while, he hid his struggles and the need to self-medicate.

Unfortunately, his combat injuries forced his retirement from law enforcement in May 2017. With little to occupy his time, the weight of his military experiences and transition out of service began to manifest more visibly in his drinking, depression, nightmares, night sweats, and negative outlook. With no badge or security clearance to worry about, he contacted the local V.A. Office. The VA diagnosed Dan with PTSD and Major Depressive Disorder (MDD) and began to treat him with Prolonged Exposure (P.E.) therapy, which he described as torturing him as much as the Trauma he had experienced. He opted not to continue this line of treatment after the V.A. canceled several appointments.

Dan went on a journey to find a better way to help himself. F ate would have Dan meet some influential folks in April 2018 at a men's leadership retreat. It was there that Dan spoke Tactical Resiliency USA into existence on Apr. 15, 2018. It has turned into an amazing organization. Tactical Resiliency USA is filled with the sincerest high contribute men and women and they are leading the charge in healing TSD, anxiety, and depression. The breakthrough in treatments with alternative non-clinical interventions were so lifechanging that they felt compelled to help others find relief from traumatic memories by sharing with the world what they found.

My training background is as a non-commissioned officer in the U.S. Army, and Drill Sergeant I focused on training groups of squads, platoons and company sized elements in Skill Level 1 and Skill Level 2 in infantry tactics.

Since 2020 I have been training the TRP, and the EMP with over 350 people trained for Tactical Resiliency USA, as well as for members of the U.S. Border Patrol, Salt Lake City Police Department, Seminole County Sheriff's Office, FL, the Lake County Sheriff's Office, FL, Alachua County Sheriff's Office, FL, the Sumter County Sheriff's Office, FL, the Broward County Sheriff's Office, FL, the Ft. Lauderdale Police Department, FL, the Clermont Police Department, FL, the Highland County Sheriff's Office, FL, Broward Fire Rescue, FL, Pompano Fire Department, FL, Gainesville Police Department, FL, the Auburndale Police Department, FL. I also have an online training platform on Kajabi which is about 4 hours' worth of work prior to the training. I am certified as an NLP Master Practitioner, NLP Practitioner, Master Hypnosis Practitioner.

I hold a Master of Science in Public Administration (MPA) from Central Michigan University, and I am currently enrolled in a Doctoral Program from Liberty University in Psychology, research not clinical track.

### Beginning the Process

TRP/EMP both begin with a conversation. The conversation alludes to a traumatic event. Within the event(s) are negative emotions: an event that causes nightmares, night terrors, flashbacks, intrusive thoughts, and isolating behaviors. The emotions are attached to the event, and this is the area that leads to the processes. Skills needed to be successful at both processes are:

### Rapport Building

Find commonalities-as the coach, listen to the individual closely to find items in common, whether cars, motorcycles, guns, sports, movies, or other common interests. In the licensed world, this is typically called the therapeutic alliance. This process will be significantly easier with peer coaches. Combat vets who have walked the same battlespace as Iraq and Afghanistan have almost instantaneous trust. Cops have walked similar beats, and firefighters who have had to save drowning children have common bonds. This process is the most important part of the protocol. It moves them to go through TRP and EMP.

Create shared experiences-attending the same events, living in the same area, shopping at the same stores, and having the same coworkers or supervisor.

Be empathetic-empathy is the ability to understand and share feelings. Empathy begins with awareness of another person's feelings and develops naturally from active listening. It would be easier to empathize if the other party told the coach how they felt. For professional counselors, this is acceptable; coaches do not get into details, and the details often trigger clients higher and make the client less comfortable, which is what they need to do to get the release. However, coaches/counselors will need to interpret nonverbal cues unless. Coaches/counselors also need to pay attention to clients' precise language.

Use active listening skills-It requires that the listener fully concentrate, understand, respond, and then remember what they are being told. To utilize active listening skills, one would reframe the last section of the last sentence into a question to clarify the individual's experience.

### Modeling

Albert Bandura is a psychologist who founded the social learning theory, which transitioned into observational learning, the origin of studies on modeling behavior-things like when a child imitates the behavior of an adult. A child sitting next to their parent has their hands clasped and legs folded over the other. The child will do the same with their body.

When an adolescent makes a face, another adolescent finds the face funny and imitates the expression. When a military service member walks with other service members, they typically start walking in step.

### Mirroring

Mirroring is the behavior in which one person subconsciously imitates the gestures, speech patterns, or attitudes of others. Mirroring often occurs in social situations, particularly in the company of close friends or family.

The concept often affects other individuals' notions about the individual exhibiting mirroring behaviors, leading to the individual building rapport with others (Eckstein, 2017). When a coach repeats two or three words of their sentence, having a conversation with someone shows them active listening takes place, typically the last two to three words.

Body language is our non-verbal communication that often says more than words. Mirroring body language is most often done subconsciously. The mirror may come as crossing or uncrossing arms and legs, but it can also be smiling, tilting the head, and leaning in when talking.

### Observing and Identifying

They were paying attention to the pulse in the neck, watching eyebrows furrow, breathing, and physiological signs of stress. Watching the eyes move behind the lids to ensure the client runs the process and knows when a person is being keyed up or shutting down—squirming in the seat, wringing the hands—paying attention to the minute changes in breath, posture, and pulse. Ears set on fire, and jaws clenched. When using telehealth, have the client set back so the coach can see the stomach up to their head. The coach can observe and identify distress.

### Break State

When clients become upset, the coach distracts them from becoming more upset. Using distractions can stop the emotion in its tracks. Disrupt the distress with a distraction or what NLP calls a “break state” (breaking the emotional state). Break state by asking them to recite their phone number backward, see the number 100 on their ceiling, look up count backward to zero (0) by sevens (7), or ask about something positive they have shared during the session.

### Exercise: Pulling Them out of Their Emotions

### Structural Components Behind Negative Emotions

Negative emotions are unpleasant or unhappy emotions evoked in clients to express a negative effect toward an event or person (Pam, 2013). If a person spends too much time dwelling on negative emotions, it becomes a spiral of rumination. Rumination tends to keep thinking, replaying, or obsessing over negative emotional situations and experiences (Nolen-Hoeksema, 1991). When the emotions affect current functioning and become intrusive on daily tasks, the EMP will become most effective.

### How the Brain Works

When an event occurs, one person may not attach any feelings to the event. Sometimes, it takes a while to realize the event coincides with emotion. The amygdala is an almond-shaped mass of gray matter inside each cerebral hemisphere involved with experiencing emotions. (Whalen & Phelps, 2009). The amygdala supports the recognition of emotions in powerful inhibitory circuits, which quiet the amygdala. The amygdala contributes significantly to anger, fear, grief, envy, and jealousy.

The data enters the amygdala; the feelings attached to the event become a trauma. The client may experience unwelcome thoughts, flashbacks, nightmares, or avoid areas where the event occurred. When these events or traumatic experiences affect a client's functioning, a mental health counselor or a psychologist will complete a PSSI5, PCL-5, or another metric to diagnose PTSD.

Strong Brain vs. Weak Brain

The subconscious mind is the most powerful part of the human mind (Rezaee & Farahian, 2015). The conscious mind holds up to 12% of the data in the brain, whereas the subconscious mind holds the remaining 88%. The conscious brain includes thinking, planning, analyzing, short-term memory, and willpower. The subconscious mind holds involuntary bodily functions, emotions, feelings, beliefs, values, habits, long-term memory, intuition, creativity, dreams, and protective responses. The Subconscious is part of the brain that can influence behavior and emotions without awareness. The Subconscious holds the information in the amygdala. The Subconscious is the part of the brain that is below our awareness. We do not see, think, or feel under anesthesia. The Subconscious implies a total lack of awareness. (Rezace & Farahian, 2015). The “strong brain” is the Subconscious; the weak brain is the Conscious part of the brain.

Brain Scan of an activated amygdala and an active Neo Cortex published by G.E. News in an article about mapping the PTSD brain.

Your body produces adrenalin when in active fight, flight, or freeze. Adrenaline is a hormone and neurotransmitter the adrenal gland produces in response to stress. It is part of the body's natural “fight or flight” response, which helps to prepare the body for physical or emotional stress by increasing heart rate, blood pressure, and blood sugar levels.

When adrenaline is released into the bloodstream, it triggers the release of glucose from the liver and fatty acids from adipose tissue. These energy sources provide the body with the fuel it need to respond to the stressor. At the same time, adrenaline also causes the air passages in the lungs to dilate, which allows for increased oxygen intake and improves breathing.

Adrenaline also has a powerful effect on the heart. It causes the heart rate to increase, which helps to pump more oxygen-rich blood to the muscles and brain. This increased blood flow allows the body to respond more quickly and efficiently to the stressor.

In addition to its physical effects, adrenaline also has psychological effects. It can cause feelings of excitement, increased alertness, and improved concentration. Adrenaline can be beneficial when quick thinking and decision-making are important, such as in sports or emergencies. However, chronic stress and sustained high levels of adrenaline can have negative effects on the body.

Constant activation of the “fight or flight” response can lead to a host of health problems, such as high blood pressure, heart disease, and type 2 diabetes. Long-term exposure to high levels of adrenaline can also increase the risk of anxiety and depression.

To reduce the negative effects of adrenaline, it is important to manage stress healthily, including regular exercise, healthy eating, and getting enough sleep. Mindfulness practices like yoga, meditation, and deep breathing can also effectively reduce stress and lower adrenaline levels. In conclusion, adrenaline is a hormone and neurotransmitter the adrenal gland produces in response to stress. It is part of the body's natural “fight or flight” response that helps to prepare the body for physical or emotional stress by increasing heart rate, blood pressure, and blood sugar levels. It provides energy and improves breathing but can negatively affect the body when the stress response is activated for a long time. Healthily managing stress and taking care of overall well-being can help reduce negative effects of adrenaline on the body.

Cortisol is a steroid hormone the adrenal gland produces in response to stress. It is part of the body's natural “fight or flight” response, which helps to prepare the body for physical or emotional stress by increasing heart rate, blood pressure, and blood sugar levels. Cortisol also plays a role in regulating metabolism, blood pressure, and the body's immune response. However, chronic stress can lead to sustained high levels of cortisol, which can negatively affect the body. Elevated cortisol levels over a long period can lead to a host of health problems, such as weight gain, high blood pressure, and an increased risk of heart disease and type 2 diabetes. High cortisol levels have also been linked to decreased bone density, which can increase the risk of osteoporosis.

Cortisol can also have negative effects on the brain. High cortisol levels over a long period can lead to decreased memory and cognitive function and an increased risk of depression and anxiety.

Chronic stress can also lead to an overproduction of cortisol, which can disrupt the delicate balance of the brain's neurotransmitters and lead to mental health problems.

Cortisol levels are also closely linked to sleep. High cortisol levels can disrupt the body's natural sleep-wake cycle, leading to insomnia, fatigue, and decreased ability to cope with stress.

To reduce the negative effects of cortisol, managing stress healthily includes regular exercise, healthy eating, and getting enough sleep. Mindfulness practices like yoga, meditation, and deep breathing can also reduce stress and lower cortisol levels.

In conclusion, cortisol is a steroid hormone the adrenal gland produces in response to stress. It plays a key role in regulating the body's “fight or flight” response; however, chronic stress can lead to sustained high levels of cortisol, which can have negative effects on the body, such as weight gain, high blood pressure, and an increased risk of heart disease and type 2 diabetes. It can also have negative effects on the brain, leading to decreased memory and cognitive function and an increased risk of depression and anxiety. Healthily managing stress and taking care of your overall well-being can help to lower cortisol levels and reduce the negative effects of cortisol on the body.

### Dissociate

Dissociation is a psychological process involving disconnection from one's thoughts, feelings, and surroundings. It is a normal response to trauma or stress but can also occur in people with certain mental health conditions.

There are several types of dissociation, including depersonalization, derealization, amnesia, and dissociative identity disorder (DID). Depersonalization occurs when a person feels detached from their body or sense of self. Derealization occurs when people feel like the world around them is not real, or things are not as they appear. Amnesia occurs when a person has gaps in memory or cannot recall certain traumatic events. DID is a rare and severe condition in which a person has two or more distinct identities or personality states.

Dissociation can be a coping mechanism that helps people to deal with traumatic or overwhelming experiences. However, if it becomes chronic, it can lead to difficulties in daily functioning and interfere with one's ability to form healthy relationships, work, or attend school. In severe cases, it can also lead to the development of mental health disorders such as PTSD, depression, and anxiety.

However, in TRP and EMP, significant healing will occur when dissociation is applied in these processes.

When a client sees the dissociated self, they can identify those features. The client appears distracted, and their reaction time is slow; they may ask the coach to repeat the instructions.

They may have pinpoint pupils, sweating, redness, signs of nervousness, or hold something back. The client could be reserved, not sharing the distress they see. Ensure that there may be signs of distress when they share what the dissociated self sees. The coach should ask specific questions, like how they reacted to the movie over there. Based on their response, do things like put the phone on airplane mode, shrink the T.V. down to the size of a cell phone, turn the T.V. off, and let the DVD run in the background.

### Dissociated Black and White Picture

The black-and-white image places the event in the past as we relate black and white to an old event or movie. The purpose behind the black and white images is to reduce the event's intensity and subconsciously indicate an old event. If the client struggles with creating the black and white images, instruct them to imagine a newspaper using dark grays, whites, and blacks; think of an old T.V. contrast knob. Make the motion to turn the contrast knob and drain the color; or an Instagram filter, or Zoom filter, the coach can show the black and white screen for remote sessions or pull out their phone and show a picture changed into black and white. The client only needs a brief image in black and white. Book Ends of the Trauma.

The start point is before the Trauma occurred when the client felt safe before Anything bad happened, the left side bookend—picture 1.

The endpoint was the right side of the traumatic event when the client was safe after the trauma was over—picture 2.

### Name or Label the Traumatic Memory

A coach can use “event” or “events”; it is benign and does not promote strong emotions. They can name the memory something else if they like. However, do not use something strong like “Slit Throat” or “College Rape.” Use Monday, January, Summertime, or something less Emotionally provoking.

### Subjective Units of Distress Scale (SUDS)

When the coach instructs the client to associate briefly with the event, the client will experience sympathetic arousal (trigger). SUDs are a scoring metric to scale the intensity of emotions in the present as the client associates them with a traumatic memory or negative emotion. The SUD scale ranges from 0-10, with 0 representing no distress and 10 representing the highest level of distress imaginable. Professionals use the scale to help individuals identify and track changes in their level of distress over time and evaluate the session's effectiveness.

### Watching the Dissociated Movie of the Trauma

The client will move to an observer position in the room, like in a doorway, looking back at themselves sitting on a couch watching a T.V. they cannot see. Please ensure they are comfortable and use olfactory, kinesthetic, or sounds to facilitate the protocol. Have them give the details of the room from recall. Describing the room opens the Subconscious even further.

They will watch the movie for 30 seconds. If the client has ADD/ADHD, shorten the movie to 10-15 seconds. Alternatively, in the observer position in the room, have them Pace back and forth. They are watching themselves sitting on a chair or couch.

### Alternatives and Options

Visual—Using visual pieces during the process is especially important during dissociation. Using favorite colors or items in the room can create an opportunity for focus. The client can turn off the T.V., and there is no sound or Picture with the DVD running in the background. (Subconscious). Make the T.V. smaller to decrease the visual spectrum, even to a cell phone on the wall.

Olfactory—The olfactory is one of the most important parts of dissociation. Olfactory—using favorite smells can minimize the impact of the event's effect. (Burning dead bodies in wartime is a smell that never leaves the mind). Having them focus on orange blossoms or hops can effect powerful changes in the dissociation.

Auditory—changing the event's sound; even though the event is running fast-forward, the event's sound exists in the back of the mind. Changing the sound to something pleasant will make the dissociation easier. They can imagine putting on their music playlist and focusing on that.

### What If/Concerns

What if the peer cannot dissociate? Have the client watch the coach or counselor as they watch their phone. The coach or counselor will watch the movie for the client and check in after each movie run.

Have the client watch a pet sleeping in the room as the pet dreams the movie from beginning to end—Check in with the pet's reactions

What if the client says they cannot find the second Picture? Help the client create a safe memory; it can be an imaginary event as long as it is after their Trauma. Often, you may have them create a still image as they look at their computer screen in the present moment.

What if the client wants their story heard? Coaches should tell them it is unnecessary to hear the story. Get them to agree to the process, and if they want to share the story after the session is closed, they will hear them out; when the emotions are stripped away, this is safer for the coach and only If the Coach is OK with this—getting them dissociated and out of the Trauma is required as quickly as possible. However, the coach is not to counsel or advise the client on the events. Instead, the coach should refer them to a licensed professional after the sessions are complete—Cognitive Behavioral Therapy, where they will be open to it.

What to do if the client gets confused about which Trauma to begin? Have the client start with the first traumatic event they recall and move forward or backward as they recall it.

What if they struggle to make the movie black and white? In black and white, they only need to see the two images briefly, pre-trauma and post-trauma. Use language for younger clients. Imagine you have that Picture on your Instagram and use the app to filter the color out. Older folks may remember the T.V. knobs for contrast, simulate turning the knob and the color draining out. These two techniques are overwhelmingly simple and effective.

When is the best time to do this process? When the client is ready for the process, that is when to start. It may be the day of the event or years after an event.

What if the client needs more help than a coach can give? Refer them to a licensed professional.

### The Rewind

The rewind is the 2-second movement backward, sounding like whoooshhh. The rewind occurs at the associated Picture 2 in black and white and back to Picture 1 in color. The rewind is part of the protocol where the neurological disconnect occurs between memory, emotion, and the amygdala. The coach or counselor looks for eye, head, or body movements to indicate that this part of the protocol has occurred. The rewind technique has also been called the Visual Kinesthetic Dissociation (VK/D), developed by Dr. Richard Bandler, a NeuroLinguistic Programming (NLP) co-founder. (Muss, D. (2002). We have researched and streamlined the process to better suit PTSD as applicable to our audience in the Trauma Resiliency Protocol.

### Reimprinting a New Emotion to the Trauma

Reimprinting of emotions is a technique used to change how one responds to a specific emotion or memory. It is based on the concept of neuroplasticity, which is the ability of the brain to change and adapt in response to new experiences. The technique changes the brain processes and stores information associated with an emotion or memory, resulting in a more positive and adaptive response. The technique is based on the idea that emotions and memories are stored in the body and mind as a neural network.

Changing how this network is activated can change how it responds to a specific emotion or memory. Reimprinting involves identifying and revisiting the memory or emotion causing distress and using various techniques to change how the brain processes and stores the information associated with that emotion or memory. Some common techniques used in reimprinting include cognitive-behavioral therapy, mindfulness, and visualization. One example of a reimprinting technique is the “Fast Phobia Cure,” which changes how the brain responds to a specific phobia. The technique involves identifying the phobic stimulus and then using visualization and other techniques to change how the brain processes and stores the information associated with that stimulus, resulting in a less intense fear response.

Reimprinting can be a powerful tool for individuals looking to overcome specific emotional responses or memories causing distress.

We are having the Client associate into a positive memory after the rewind decreases the SUDs to 3 or less. The associated positive emotion should bring the emotions down to a SUDs 0. If there is still a low SUD number, determine the nature of the emotions, use EMP, and the remainder should be released.

### Grounding After a Session

Diaphragmatic breath exercise-start with a deep four-second inhale, hold the inhale for four seconds, exhale for six seconds, and compress the belly to the spine with a four-second hold at the bottom of the exhale. Repeat this for five repetitions.

Alternate Grounding—“Imagine your feet are tree roots growing 100 feet into the earth, slap your legs, rub your arms, breathe in through your nose and out through your mouth enough times you are calm and fully inside your body.”

Alternate Observers—The clients can observe themselves watching the movie. If the client has self-esteem issues and do not want to watch themselves, they can put someone else in the chair, like a superhero, a grandparent, a best friend, or even a pet.

Anxious Clients—Have the Client hold their breath for 10 seconds in the 30-second window of the dissociated movie.

Field of View—Have the Client shrink the T.V. to a cell phone to help dissociate the movie.

Turn Off the T.V. Give the client control to turn off the T.V. and instruct them that the DVD will run only in the background.

### Emotions Management Process (EMP)

Negative Emotions:

Rage, Frustration, Anger, sadness, fear, shame, hurt, guilt, survivor guilt abandonment, disgust, panic, anxiety, limiting belief structures, self-worth, disgust, unforgiveness, bitterness, isolation, loneliness, hopelessness, rejection, resentment, etc.

People learn and process emotional patterns from their life experiences. The imprinting phase of learning is the first 6-7 years of life. Children soak up information like a sponge and develop patterns of behavior that suit their needs. A child gets fed when they cry, or their diapers are changed. A child witnessing a parent reacting with emotions will reinforce that child's behavior to act the same. It's why families tend to experience similar experiences of the parent(s) or cycles of behavior.

Belief structures are deeply rooted in the subconscious brain, from repetition learning throughout your life, and we are creatures of habit. Take politics; kids tend to follow the people they admire and respect. If mom or dad is revered, the child has little choice until the child grows up, and his or her paradigm is challenged, and they change because of a core belief structure.

You can change the input and build resiliency beyond your imagination. If a coach tells a football player that he will never amount to Anything often, the kid takes it to heart and gives up on a dream. Or a mother tells her daughter she is worthless and then she believes it. The EMP process focuses on using your voice to repair past damage. “You are your greatest human authority.”

### Make Sense Mandate

The human brain needs to make sense of the data that comes into the brain, commonly referred to as “the make senses mandate.” for the EMP process, we can go before the event subconsciously. The brain understands the event with its current knowledge, resulting in the release of unhealthy negative emotions. At the same time, it's in full control, and they will reframe the emotions with new learnings.

You being your biggest authority is the best way to make this happen. Get the client in the strong brain, the “Subconscious brain.”

The first step is to get the client into a subconscious memory. Have the client you are working with remember a past positive event that was enjoyable for them. Have them go into the event in their mind to feel the emotions and recall as much detail as possible, including sounds, smells, or tastes. (This is typically only necessary when working with the first set of negative emotions because you have already opened that part of the subconscious brain.) A memory that opens the imagination and memory recall will have a favorable effect.

The Creative Subconscious is Powerful. Use it.

Be creative with the Subconscious; it cannot tell the difference between what it imagines, experiences, or dreams. For example: Civilian Client

Client: “I feel stuck in a hole and can't get out.” Practitioner: What if you gave the younger self a ladder, a rope, and a friend at the top? Imagine that taking place. SUDs went from 10-0.

### Veteran Client

Client: “I can't let go of my anger; my dad deserves it.” SUDs were a 10. Practitioner: “What is your dad (retired Green Beret Colonel) doing now?”

Client: “That asshole is retired, getting 100 percent on top from the V.A. working a GS13 job. He's doing whatever the Fu@$ he wants.”

Practitioner: “Then why the hell are you carrying his ruck?”

Client: “Holy $hi7, what am I carrying it for?” SUDs 10-0

### Veteran Client

Client: “I feel like I should feel guilty because I'm alive, and they aren't.”

Practitioner: I want you to trade places with them. You aren't going to survive, and they are. What do you want for their life?”

Client: “I want him to be happy, have a family, and live their life to the fullest.”

Practitioner: “What makes you think they expect less from you?”

Client SUDs dropped from 10-4.

Practitioner: “Imagine your squad is taking your ruck from you after the IED blast; the ruck is full of all your survival guilt. They are taking it a safe distance, and you watch them prep the ruck with C4, and they move to a safe distance. You hear your friend yell out,

“Fire in the hole, fire in the hole, fire in the hole,” and then boom, you see the explosion as it destroys your ruck with your remaining survivor guilt.” Client SUDs dropped from 4-0.

### Firefighter Client

Client: “I feel I will always feel fear and panic.” The client remembered his first panic attack, associated it with the event, and triggered it to a 10. He reframes using spiritual and biblical verses because he believed in the Christian Faith.

Practitioner: “What does the bible say about fear and panic?”

Client: The Client quoted chapter and verse and paused with a look of surprise, and his ten (10) went to a 0.6 months later, a check-in revealed no more panic attacks, no more medications, and been on three family vacations. Higher power is more effective than self-power. It does not matter what that power is as long as they acknowledge it.

### REFERENCES

Global Post Traumatic Stress Injury Foundation. (2010). Retrieved on Jun. 20, 2020 from: http://globalptsifoundation.org/Mehrdad
Rezaee, Majid Farahian. Subconscious Vs. Unconscious Learning: A Short Review of the Terms. American Journal of Psychology and Behavioral Sciences. Vol. 2, Nov. 3, 2015, pp. 98-100.
Whalen, Paul, J., Phelps, Elizabeth, A., (2009). The human amygdala 1st ed., The Guilford Press, New York, London, Found online at: https://epdf.pub/the-human-amygdala.html
Muss, D. (2002). The Rewind Technique in the treatment of posttraumatic stress disorder: Methods and Application Brief Treatments for the Traumatized. C. R. Figley. West Port, Conn, Greenwood Press: 306-314.
The Power of the Subconscious Mind” by Dr. Joseph Murphy
The Role of the Subconscious in Creativity” by Psychology Today
The Subconscious Mind: How to Access and Use It” by the American Institute of Stress
The Power of the Subconscious Mind: How to Use it for Success and Happiness” by Dr. Bruce Lipton
“The Subconscious Mind” by the American Psychological Association
The subconscious mind and how it affects behavior” by the American Psychological Association Subconscious
Processing: How the Unconscious Mind Influences Decision Making.” by J. Scott Armstrong in the Journal of Marketing Research
The Power of the Subconscious Mind: How to Use Its Techniques for Personal Growth” by Joseph Murphy, Ph.D.
“The Rewind Technique for PTSD” by the National Center for PTSD
The Rewind Technique for Trauma” by the British Association for Behavioral and Cognitive Psychotherapies (BABCP).
The Rewind Technique: A New Approach to Trauma Therapy” by the Trauma Recovery Center
The Rewind Technique: An Overview” by EMDR Europe
Cortisol: The Stress Hormone” by Harvard Health Publishing, published by Harvard Medical School, 2020.
The effects of cortisol on the body” by Dr. Joseph Mercola, published by Mercola, 2020
Cortisol: The Good, the Bad, and the Ugly” by Dr. Laura L. Smith, MD, published by Psychology Today, 2019.
Adrenaline by MedlinePlus, published by the U.S. National Library of Medicine, 2021
Adrenaline: How the ‘fight or flight’ hormone affects your body” by Dr. Anand Patel, published by Medical News Today, 2019
Adrenaline: The Good, the Bad, and the Ugly” by Dr. Laura L. Smith, MD, published by Psychology Today, 2019.
The Trauma Resiliency Protocol (For non-visual clients, add a practice movie)

### Phase One: the Trigger & Extraction of the Traumatic Feeling

Words spoken to the client are in bold.

Coach notes are in italics. These words are for the coach/counselor only, not told to the client.

Rapport Building First and then Step 1:
A. Think about the event causing you distress. Please tell me if you trigger it, and I do not see it. If I see that you are triggered—becoming anxious or upset—we will take a break to resettle. We need you to be calm after the initial trigger. Go ahead and think of the event as if you are there again; do not share any details.
   Observe the client with sensory acuity. The client does not relate Anything about the traumatic event to the coach. That is not necessary for healing to occur.
   If you see signs that the client has triggered, and you see things such as trembling hands, quivering lips, heavy breathing, rapid eye movement, etc., pause the exercise. Ask the client to look up and recite their phone number backward or count backward from 100 by 7's. Break state as needed.
B. Where are you now on a scale of zero to ten, with zero being no negative emotions and ten being extraordinarily strong negative emotions relating to fear, terror, and helplessness? [symbol]
C. What do you want to call this memory? [symbol]
   BREAK STATE (Be prepared to do this quickly and repeatedly if
D. Create a starting point before the event where you were safe before Anything bad happened. (Pause) When you have the image, drain the color so it is black and white. What is Picture 1?
   Picture 1

[symbol]

You can open multiple triggers and expand your bookends to cover more of a longer period. For example, a 30-year career in Law Enforcement; Picture 1 is the day he was first sworn in as an officer; Picture 2 is the day the officer retired. Activate numerous triggers with a break state and SUDs for each event. Opening twenty traumatic triggers and disconnecting them all in a single session is possible but be cautious based on your skill level.
E. Now, create an ending point when the event is finished, and you are safe; again, drain the color and make it black and white. What is Picture 2?
   Picture 2

[symbol]

Words spoken to the client are in bold. Coach notes are in italics. These words are for the coach/counselor only, not told to the client.
F. Make your movie environment as comfortable as possible: Describe your room, what you are watching, smelling, etc. (man-cave, bedroom, basement, cell phone, tablet, T.V. size). Have them smell popcorn, taste beer, or listen to music. Kinesthetic feelers may want the A.C. at 65 wrapped in a heavy blanket with a dog on their lap; auditory people may want background music; visual people may want dimmed lights, etc.
G. Now, sit on your sofa or chair and mentally place [symbol] Picture 1 in black and white on your screen. Is it there? [symbol]
H. Now remain seated on the sofa/chair and float to the observer position, somewhere you can't see what's on the TV screen. The other you will remain over there to watch the movie from the sofa/chair. In the observer position, you will only watch the other you on the sofa/chair as they are watching the movie. Understand? [symbol]
I. You in the sofa/chair are going to watch your movie in black and white, starting at [symbol] Picture 1 and ending at [symbol] Picture and will take only 30 seconds. After 30 seconds, I will say when you are done.
   Are you ready? Go. (3-5 reps) After 30 seconds, stop them if they are still watching them and check-in.
J. How was that for you in the observer position? [symbol] What were you in the sofa/chair doing while they watched the movie? [symbol] (repeat lines 9-10 up to 5 times)
   Do not move on until the client is comfortable with this step. Repeat as needed. (A minimum of three observations of the dissociated movie should take place, but as many as necessary.) Add adjustments if necessary, such as Shrinking the T.V. to the size of a cell phone, making it more difficult to see and hear.—“Since it's your movie, tell yourself to pick up the remote, turn the picture, and sound off; the DVD will just run in the background.”
   I will tell you during the 30-second movie to hold your breath for 10 seconds at some point; when I say breathe, keep watching yourself over there.”
K. You will step into the end of your movie as yourself in Picture 2; instead of watching it, you will be in the scene reliving the experience. There will only be you in the movie's last scene, as you see it through your own eyes and hear it through your ears, fully associated in black and white. Do you understand? After they are associated, have them signal when they are ready to run the movie backward and associate the last scene in Picture 2, black and white, with the first scene in Picture 1, in color.
   Words spoken to the client are in bold. Coach notes are in italics. These words are for the coach/counselor only, not told to the client.
L. From inside the end of the movie imagine being associated in Picture 2 in black and white. In a moment, you are going to move backward, from the end of the movie, in only two seconds, undoing all the actions of the event to the beginning of the movie, where it ends in color, where you are safe before anything bad happens. Think of fast-forwarding through a video but in reverse. You will stop at Picture 1 as it transitions back into color. As you start, I will say, “Whoooshhh,” and—just that fast—relive the entire movie backward. Have you got it? Think of walking backward, running backward, and moving through a wind tunnel to the beginning, undoing all the actions quickly. UNDERSTAND? Repeat instructions as needed. Do this a minimum of three times.
M. Please give me a thumbs up or nod when you are in the end of your movie and ready to move backwards. (Pause) Here we go.

Whoooshhh! Draw out “whoooshhh” for two seconds.
N. Did you make it back to Picture 1? [symbol] Did picture one transition back to color? [symbol]
   Repeat until the process is completed easily by the client. Run this rewind (Line 13) 3-4 times, but as many as necessary.
O. Think about the event that has been causing you distress again, and on a scale from zero to ten, with zero being no negative emotions and ten being extraordinarily strong negative emotions relating to fear, terror, and helplessness, where are you now?

[symbol]

If the Client's Subjective Units of Distress (SUDs) are three or below, they are ready for Phase Two. If the SUDs are four or above, return to Step 8 and repeat until the score is three or fewer.

If running multiple traumas, check the SUDs of each event. The same rules apply to three or fewer resets in the move to phase 2, four (4) or more, back to the dissociated movie at line 8, and continue.

Words spoken to the client are in bold. Coach notes are in italics. These words are for the coach/counselor only, not told to the client.

### Phase Two—Resetting the Emotion

P. Now, you will think of a more satisfying event where you remember feeling joy, happiness, excitement, or other positive feelings; go ahead and do it now. Pause to give the Client time to recall a different, more positive event.
Q. Focus on the emotions of that better event for around a minute; feel the joy, happiness, and excitement, and focus on feeling the emotions again as you relive the experience. Can you feel it? Now, imagine a dial in the lower right corner; turn it up 10 times more intensely and enjoy the moment. Pause and ensure they can feel the emotions of the good memory for a minute before moving on.
R. Think about the event that has been causing you distress again, and on a scale from zero to ten, with zero being no negative emotions and ten being extraordinarily strong negative emotions relating to fear, terror, and helplessness, where are you now? [symbol] (If there is still a residual SUDS after line 18, then transition to EMP, label the emotion(s), and EMP the residual SUDs to get it to 0)

Do a grounding exercise if they feel they are still floating around and dissociated.

Breath work can work well for this; imagine sitting on their lap, drawing back into their body as they do deep diaphragmatic breathing.

### The TRAUMA Resiliency Protocol-Jesus Protocol (For Non-Visual Clients, add a Practice Movie)

PHASE 1: Elicitation then Extraction of the Traumatic Emotions. Words spoken to the client are in bold. Coach notes are in italics. These words are for the coach only, not told to the client.

Rapport Building First and then Step 1
A. Think about the event causing you distress. Please tell me if you trigger it, and I do not see it. If I see that you are triggered—becoming anxious or upset—we will take a break to resettle. We need you to be calm after the initial trigger. Go ahead and think of the event as if you are there again; do not share any details.
   Observe the client with sensory acuity. The client does not relate Anything about the traumatic event to the coach. That is not necessary for healing to occur.
   If you see signs that the client has triggered, and you see things such as trembling hands, quivering lips, heavy breathing, rapid eye movement, etc., pause the exercise. Ask the client to look up and recite their phone number backward or count backward from 100 by 7's. Break state as needed.
B. Where are you now on a scale of zero to ten, with zero being no negative emotions and ten being extraordinarily strong negative emotions relating to fear, terror, and helplessness? [symbol] BREAK STATE (Be prepared to do this quickly and repeatedly if necessary.)
C. What do you want to call this memory?
D. What were you doing when you were safe before the event?

(Pause)

[symbol]

Make a picture of the safe spot before the event and drain the color so it is black and white. Is that done?

[symbol]

You can open multiple emotional triggers and expand your bookends to cover more of a longer period. For example, a 30-year career in Law Enforcement; Picture 1 is the day he was first sworn in as an officer; Picture 2 is the day the officer retired. Activate numerous triggers with a break state and SUDs for each event. Opening twenty traumatic triggers and disconnecting them all in a single session is possible but be cautious based on your skill level.

Words spoken to the client are in bold.

Coach notes are in italics. These words are for the coach only, not told to the client.
E. What were you doing when the event ended and was over, and you were safe?
   (Pause)

[symbol]

Please take a picture of the safe spot after the event ends and drain the color so it is black and white. Is that done?
F. Now, imagine you are sitting in a room with Jesus and watching Him (mancave, bedroom, basement, or Jerusalem watching a cell phone, tablet, T.V. size). Have them smell popcorn or hear angelic music. Kinesthetic feelers may want the place cold at 65, wrapped in a heavy blanket with a dog on their lap; auditory people may want background music; visual people may want dimmed lights, etc.
G. Jesus will bring up your movie in front of Him; you initially see the first Picture in black and white; however, as you stand off in the corner and observe Jesus, He then wraps a light cloud around the movie, not allowing you to see or hear what is going on from inside the movie as it plays from beginning to end.
H. Jesus will watch your movie for you; you know He is stronger than anyone; He can handle it. As the observer, you will only watch Jesus as He watches your movie, understand? [symbol]
I. Jesus will now watch your movie in black and white, starting at [symbol] Picture 1 and ending at [symbol] Picture 2, taking Him only 30 seconds; after 30 seconds,
   I will say, when you are done watching Jesus. Are you ready? Go.

After 30 seconds, stop them if they are still watching them and check-in. How was that for you? How was that for Jesus?
J. What was Jesus doing while He watched your movie? How was that for you observing Jesus? [symbol] Make this observation pattern occur 3-5 times.

Do not move on until the client is comfortable with this step. Repeat as needed.

(At least three observations of the dissociated movie should occur, but as many as necessary.) Add adjustments if necessary, such as shrinking the T.V. to the size of a cell phone or a stamp and turning it off, making it more difficult to see and hear.

“Since it's your movie, tell yourself to pick up the remote, turn the picture, and sound off; the DVD will just run in the background.”

Words spoken to the client are in bold.

Coach notes are in italics. These words are for the coach only, not told to the client.

I will tell you during the 30-second movie to hold your breath for 10 seconds at some point; when I say breathe, keep watching yourself over there.
K. You will step into the end of your movie as yourself in Picture 2; instead of watching it, you will be in the scene reliving the experience. There will only be you in the movie's last scene, as you see it through your own eyes and hear it through your ears, fully associated in black and white. Do you understand? After they are associated, let them signal when they are ready to run the movie backward and associate the last scene in Picture 2, black and white, with the first scene in Picture 1, in color.
L. From inside the end of the movie imagine being associated in Picture 2 in black and white. In a moment, you are going to move backward, from the end of the movie, in only two seconds, undoing all the actions of the event to the beginning of the movie, where it ends in color, where you are safe before anything bad happens. Think of fast-forwarding through a video but in reverse. You will stop at Picture 1 as it transitions back into color. As you start, I will say, “Whoooshhh,” and—just that fast—relive the entire movie backward. Have you got it? Think of walking backward, running backward, and moving through a wind tunnel to the beginning, undoing all the actions quickly. UNDERSTAND? Repeat instructions as needed. Do this a minimum of three times.
M. Please give me a thumbs up or nod when you are in the end of your movie and ready to move backwards. (Pause) Here we go.

Whoooshhh! Draw out “whoooshhh” for two seconds. Do this 3-5 times before moving to 15.
N. Did you make it back to Picture 1? [symbol]
   Did Picture 1 transition back to col

[symbol]

Repeat until the process is completed easily by the client. Run this rewind at least 3-5 times, but as many as necessary.
O. Go ahead and think about the event that has been causing you distress again, and on a scale from zero (0) to 10, with zero (0) being no traumatic emotions and 10 being extraordinarily strong traumatic emotions relating to fear, terror, and helplessness, where are you now?

[symbol]

If the Client's Subjective Units of Distress (SUDs) are three or below, they are ready for Phase Two. If the SUDs are at four (4) or above, return to Step 8 and repeat until the score is three or fewer.—If running multiple traumas, check the SUDs of each event. The same rules apply: three (3) or fewer reset in the move to phase 2, 4, or more, move back to the dissociated movie at line 7, and continue.

Words spoken to the client are in bold.

Coach notes are in italics. These words are for the coach only, not told to the client.

### Phase Two—Resetting the Emotion

P. Now, you will think of a more satisfying event where you remember feeling great joy, happiness, and excitement, perhaps at your baptism or when you decided to accept Christ for the first time.

Pause to give the Client time to recall a different, more positive event.
Q. Can you feel positive feelings now? Focus on the emotions of that better event for around a minute; feel the joy, happiness, and excitement, and focus on feeling the emotions again as you relive the experience. Good. Now imagine it gets brighter and brighter and more focused; the feelings grow more intense as this happens. Sit there for a moment and enjoy the feelings.

Pause and ensure they can feel the emotions of the good memory before moving on.
R. Think about the event that has been causing you distress again. Where are you now on a scale from zero (0) to 10, with zero being no traumatic emotions and 10 being extraordinarily strong traumatic emotions relating to fear, terror, and helplessness?

[symbol]

(If there is still a residual SUDS after line 18, then transition to EMP, label the emotion(s), and EMP the residual SUDS to get it to 0.)

Do a grounding exercise if they feel they are still floating around and dissociated.

Breathwork can work well for this exercise.

### The Emotions Management Process (EMP)

Common Negative Emotions: Rage, Anger, sadness, (do in same session) frustration, rejection, criticisms, self-sabotage, hopelessness, fear, worry, anxiety, hurt, guilt, survivor guilt, shame, money fear, abandonment, resentment, disgust, bitterness, unforgiveness, and any self-limiting decision or belief.

Vantage Points: You are floating above a drone, a hot air balloon, a helicopter hovering above, SRV71 in the outer atmosphere, etc. Be creative; allow the client to assist in this. It empowers them. If they cannot see dissociated like that, have them go to an event in their memory where they can feel the negative emotion, then manipulate the screen from a visual and kinesthetic viewpoint to get it disconnected.
A. Return to the moment of your birth; let me know when you are there. Imagine someone holding you that would be safe for you. Are you there? [symbol]
B. Move forward to the first time you could feel the emotion of: [symbol] Drop into the moment seeing what you saw, hearing what you heard and feeling what you felt at the time and let me know if you can feel it. How intense does it feel, with zero (0) being no emotions and ten (10) being the most intense as you feel it in your body right now? [symbol]
C. What do you want to call this memory? [symbol] and how old are you approximately?

[symbol]

Break State

Note: (Anger-Sadness-Fear-Shame-Guilt-Hurt-Abandonment-Rage-Hate-Anxiety-Panic or a self-limiting belief etc.)

Note: Allow them to associate with the memory to feel the emotion; if they cannot feel it at present, they have already processed and released the emotion and moved to another event.
D. Return to your birth; let me know when you are there. Can you feel the old emotion of [symbol] at birth, or perhaps not?

[symbol]

Note: You can use language like, obviously, at age three, you cannot know how to process emotion. Perhaps someone in your home was (negative emotion), etc. (Anger, sadness, fear, shame, hurt, etc.)
E. You have had some experiences since the first time you felt the emotion of [symbol] . What would you tell your younger self, the learnings of which would allow you to learn what you need from the event but quickly, easily, and effortlessly let go of the negative emotion of [symbol] , but preserve the learnings for future use instead of the emotions?

Note: (The new learnings must be positive, self-focused, and future focused. “It was not to blame; I'm going to be OK. I am still loved. I am worthy as I am, etc.

[symbol]

Note: (If they still feel the negative emotion before the first event, there may be an earlier event; try and find the earliest event or take them back to the moment of birth and have them imagine being in their mothers' arms, but only if that is a safe place. If mom is not safe, take them to the moment of conception and imagine being a single-cell organism before anyone knew they existed.)

F. Listen to my instructions before starting. In a moment, you will move forward from birth and move to just before the first event of [symbol] and be able to give yourself new learnings. (Repeat the learnings for them.) Look at the events differently and reframe how you understand the event with today's new learnings. You will then quickly, easily and effortlessly let go of the negative emotion of [symbol] and move to the next event in your life where you can feel that same negative emotion, stopping at each event, re-frame each event, move forward looking for all the events, release all negative emotions, and return to the present only as soon as you are ready. Do you understand? OK Go.

Note: When they are back to the present, do a BREAK STATE

(BREAK STATE: From what school did you graduate? What are the last four of your phone numbers backward? Anything to change their emotional state. You are getting them off-topic briefly.)
G. Test: (Individual is back at present.) Can you remember that event when you used to feel that old negative emotion? Go back and notice if you can feel it, or you may find that you may not feel it. Good, come back to the present. (If they still feel it.) How would you rate the intensity of emotion on a 0-10 scale, with 10 being the most intense? SUDS:

[symbol]

Note: Test until the individual is convinced. (Take them back to the initial memory where they reported a SUDS. See if they can feel it at that moment.) If they cannot feel it, move to line 10. If they still feel it, there may be additional learnings or a different event altogether. Go back to the event, go back to line 4 and continue.
H. Future Pace: (The individual is back at present.) Please go out into the Future to an unknown date or time when, in the past, you would have felt inappropriate or unwarranted negative emotions of [symbol] . Note: (name the emotion) and notice if you can feel that old emotion as the event unfolds, or you may find that you cannot feel it. OK? [symbol] (if 0 you are done) If you can still feel it move two weeks into the future after it is gone and turn around and look back from the future, are you there? [symbol] . (Reverse Learnings from the future)
I. What would the older self-teach the younger self the learnings of which would allow the emotion of [symbol] quickly, easily, and effortlessly, fall away, but preserve the learnings since they are useful, the emotions are not. [symbol]
J. Move from the future backwards through the event two weeks from now and apply the learnings so you can quickly, easily and effortlessly let go of the emotion of [symbol] and then move back through the present all the way to birth looking for the emotion of [symbol] and re-framing how you look at each event releasing the emotion of [symbol] . Are you back at birth? [symbol] Now move forward looking for any emotions of [symbol] that you may have missed back to the present.
K. Now move back to that future event where you felt the emotion of [symbol] can you feel it now or perhaps not? [symbol] Good. The emotion should be 0 in the future at this point.

Note: Also, test how the situation plays out in the Future. Is it different than the past that would have unfolded? Note: If the emotions do not disappear, then reframe again.

If they no longer feel the emotion, move to the next emotion with line number 2.

There are three things to check on the hover position if they still feel the old emotion.

Make sure the individual is now up above the event.

(Indicator: Individual is feeling the emotions)

Tell them:

Move up higher, above the event, maybe with a birds-eye view or cruising altitude of a 747, just high enough not to feel the emotions; this will typically sever the emotions.

Get up high enough until the emotions disappear.

Be sure the individual is present before the first event. (Indicator: The bulk of the emotions released.) Ask the individual:

Are you before the first event?

Is there any event that is even earlier than this one? Go back to before the FIRST one.

If the client does not release the emotion, you must discover why they do not want to let it go. They must be fully agreeable to letting go of this emotion. There may be a secondary gain for them to hold it. Reframe and dispute the gain.

(Indicator: The individual says, “The emotions are still there.”) Ask them:

What is there to learn from this event? If you learn this, won't it be better than having the old emotions? How can you get the same benefit that the emotions provide when you let them go?

(Use this reframe or any other reframe on GENERAL REFRAMES)

### Notes Regarding Learnings

Learnings are not always obvious to the individual. Especially where the individual already has learnings, they may not get any new or obvious ones. When you (as the coach) are aware of learnings, they should NOT be Negative in the past and rely on Others.

When you know about learnings, they should be positive, Self-focused, and future-focused.

Note: If the individual still feels the emotions, have them float above their timeline to the event where they initiate a SUD response. Ask them whether it is a significant emotional event or a traumatic event. If they say the significant emotional event, test it with a subjective unit of distress scale for terror, fear, and helplessness on a scale of 0-10. [symbol] If five or more execute the Trauma Resiliency Protocol (TRP) on that event, it is more than likely a trauma. And if not, it is OK to get that 5-0.

General Reframes

USE General reframes if the NEGATIVE

EMOTIONS DO NOT RELEASE BASED UPON:

Learning. What is there to learn from this event, the learnings of which will allow you to let go of the emotions with little effort easily and quickly? Won't it be better to preserve the learnings rather than the emotions? If you let go of the emotions and preserve the learnings, you will have learned what you need.
a) Protection/Safety:

“The negative emotion of doesn't protect you.”

(If you are working with fear or Anger, mention flight or fight) Negative emotions are not safe for the body.

Each negative emotion can contribute to any number of types of health problems:

Diabetes, heart disease, strokes, heart attacks, alcoholism, pick your cancer, obesity, etc.

Won't you be much safer if you let go of the emotions and preserve the learnings about taking care of yourself?”
b) Brain self-preservation:

Not letting go of this emotion directly conflicts with the highest purpose of the subconscious mind, which is, ‘To preserve the body.’ Though it is getting results, this emotion does not preserve the body; it hurts it. Wouldn't it be better to let go of the repressed emotion and get the same results in another way?

The Emotions Management Process (EMP) with JESUS Notes to the Coach are in Italics and are Not to be Spoken

Words spoken are in bold. Common Negative Emotions

Rage, Anger, sadness, frustration, rejection, criticisms, self-sabotage, hopelessness, fear, worry, anxiety, hurt, guilt, survivor guilt, shame, money fear, abandonment, resentment, disgust, bitterness, unforgiveness, and any self-limiting decision or belief.

Vantage Points: You are floating above a drone, a hot air balloon, a helicopter hovering above, SRV71 in the outer atmosphere, etc. Be creative; allow the client to assist in this. It empowers them. If they can't see dissociated like that, have them go to an event in their memory where they can feel the negative emotion, then manipulate the screen from a visual and kinesthetic viewpoint to get it disconnected.
a) I want you to imagine you are floating in the clouds with Jesus; He's showing you your life, floating over your timeline of events. He will take you back to the moment of your birth; let me know when you are there where it all began for you.

Note: (Anger-Sadness-Fear-Shame-Guilt-Hurt-Abandonment-Rage-Hate-Anxiety-Panic, disappointment, frustration, rejection or a self-limiting belief, etc.)
b) Imagine Jesus walking forward with you from birth and finding the first time you felt the negative emotion of how old you are approximately.
c) Drop into the memory and see what you saw, hear what you heard, and feel what you felt at the time, but know Jesus is with you to protect you. How would you rate the intensity of emotion on a 0-10 scale (SUDS), with 10 being the most intense as you feel it now?

Note: allow them to associate with the memory to feel the emotion; if they cannot feel it, they have already processed and released the emotion. Move them to another event.
d) Now move back to the moment of birth with Jesus, before the emotion of, let me know when you are back there. Do you feel the old emotion of or perhaps not?

Note: You can use language like, obviously, at age three, you cannot know how to process emotion. Perhaps someone in your home was (negative emotion), etc. (Anger, sadness, fear, shame, hurt, etc.)

You have had some life and faith experiences since the first time you felt the emotion of. Jesus will now tell you some learnings out of His word, the learnings of which will allow you to easily, effortlessly, and quickly let go of the emotion of but preserve the learnings so you can use the information in the Future. (Bible Verses)

Note: (The new learnings must be positive, self-focused, and future-focused. “I was not to blame; I′m going to be OK. I am still loved. I am worthy as I am, etc.”)
e) Jesus will now take you back to your past event where you first felt the emotion of and apply your new learnings, look at them differently, and let the emotions fall away easily, effortlessly, and quickly, then move forward to every other event where is present back to the present, letting the emotions fall away, for they don't serve you. BREAK STATE

Note: (If they still feel the negative emotion before the first event, there may be an earlier event; try and find the earliest event or take them back to the moment of birth and have them imagine being in their mothers' arms, but only if that is a safe place. If mom is not safe, take them to the moment of conception and imagine being a single-cell organism before anyone knew they existed.)

Note: When they are back to the present, do a BREAK STATE.

(BREAK STATE: From what school did you graduate? What are the last four of your phone numbers backward? Anything to change their emotional state. You are getting them off-topic briefly.)
f) Test: (Individual is back at present.) Can you remember that event when you used to feel that old negative emotion? Go back and notice if you can feel it, or you may find that you may not feel it. Good, come back to the present. How would you rate the intensity of emotion on a 0-10 scale, with 10 being the most intense? SUDS:

[symbol]

Note: Test until the individual is convinced. (Take them back to the initial memory where they reported a SUDS. See if they can feel it at that moment.) If they cannot feel it, move to line 10. If they still feel it, there may be additional learnings or a different event altogether. Go back to the event, go back to line 4 and continue.
g) Future Pace: (The individual is back at present.) Go out into the Future to an unknown date or time when you would have felt inappropriate or unwarranted negative emotions in the past. Note: (name the emotion) and notice if you can find that old emotion as the event unfolds, or you may find that you cannot find it. OK?

Note: Also, test how the situation plays out in the Future. Is it different than the past that would have unfolded?

Note: If the emotions do not disappear, then reframe again.

If they no longer feel the emotion, move to the next emotion with line number 2.

If they still feel the old emotion, there are three things to check on the hover position.

Make sure the individual is now up above the event.

(Indicator: Individual is feeling the emotions) Tell them:

Move up higher, above the event, maybe with a birds-eye view or cruising altitude of a 747, just high enough not to feel the emotions; this will typically sever the emotions.

Get up high enough until the emotions disappear.

Be sure the individual is present before the first event. (Indicator: The bulk of the emotions released.) Ask the individual:

Are you before the first event?

Is there any event that is even earlier than this one? Go back to before the FIRST one.

If the client does not release the emotion, you must discover why they do not want to let it go. They must be fully agreeable to letting go of this emotion. There may be a secondary gain for them to hold it. Reframe and dispute the gain.

(Indicator: The individual says, “The emotions are still there.”) Ask them:

What is there to learn from this event? If you learn this, won't it be better than having the old emotions? How can you get the same benefit that the emotions provide when you let them go?

(Use this reframe or any other reframe on GENERAL REFRAMES)

### Notes Regarding Learnings

Learnings are not always obvious to the individual. Especially where the individual already has learnings, they may not get any new or obvious ones. When you (as the coach) are aware of learnings, they should NOT be Negative in the past and rely on Others.

When you know about learnings, they should be positive, Self-focused, and future focused.

Note: If the individual still feels the emotions, have them float above their timeline to the event where they initiate a SUD response. Ask them whether it is a significant emotional event or a traumatic event. If they say the significant emotional event, test it with a subjective unit of distress scale for terror, fear, and helplessness on a scale of 0-10. [symbol] If five or more execute the Trauma Resiliency Protocol (TRP) on that event, it is more than likely a trauma. And if not, it is OK to get that 5-0.

### General Reframes

USE WHEN NEGATIVE EMOTIONS DO NOT RELEASE BASED UPON: Learning

What is there to learn from this event, which will allow you to let go of emotions with little effort easily and quickly? Won't it be better to preserve the learnings rather than the emotions? If you let go of the emotions and preserve the learnings, you will have learned what you need.

Protection/Safety:

“The negative emotion of doesn't protect you.”

(If you are working with fear or Anger, mention flight or fight) Negative emotions are not safe for the body.

Each negative emotion can contribute to various health problems: Diabetes, heart disease, strokes, heart attacks, alcoholism, pick your cancer, obesity, etc.

Won't you be safer if you let go of the emotions and preserve the learnings about taking care of yourself?”

Brain self-preservation:

Not letting go of this emotion directly conflicts with the highest purpose of the subconscious mind, which is, ‘To preserve the body.’ Though it is getting results, this emotion does not preserve the body; it hurts it. Wouldn't it be better to let go of the repressed emotion and get the same results in another way?

The Positive TRAUMA Protocol-Secular For Pride and Narcissism

PHASE 1: Elicitation then Extraction of the Positive Trauma Emotions

Words spoken to the Client are in bold.

Coach notes are in italics. These words are for the coach only, not told to the Client.

Rapport building first and then step 1:1) I want you to imagine you are on your sofa watching your TV. Can you see that?

[symbol]

It is time to think about the first event causing you elevated excitement or positive emotions due to elevated status or recognition. You can, associate with the event, and even talk about it to really get the positive feelings, imagine being there again seeing what you saw, hearing what you heard and feeling what you felt at the time.

Observe the Client with sensory acuity. Break state as needed.

As you think about the event, where are you now on a scale of 0 to 10, with 0 being no excited or positive emotions and 10 being extraordinarily strong excited or positive emotions relating to an elevation of status?

What do you want to call this memory?

### Break State

What were you doing when you were before the event occurred? (pause)

Make a picture of the safe spot before the event and drain the color so it is in black and white. Is that done?

Words spoken to the Client are in bold.

Coach notes are in italics. These words are for the coach only, not told to the Client.

You can open multiple emotional excited positive triggers and expand your bookends to cover more of a longer period. For example, a 30-year career in Law Enforcement; Picture 1 is the day he was first sworn in as an officer; Picture 2 is the day the officer retired. Activate numerous excited triggers with a break state and SUES for each event. Opening twenty excited triggers and disconnecting them all in a single session is possible, since these are safe moments and times there is limited concern unless you inadvertently open traumatic regressors. If so, break state and include the SUDS.

What were you doing when the event ended, and you were safe? (Pause)

Make a picture of the safe spot after the event ended and drain the color so it is in black and white. Is that done?

[symbol]

As you are seated on your sofa or chair place the first picture of you [symbol] [symbol] before your event where you were safe before anything happened up on your screen. (This can be any room like a man-cave, bedroom, basement, cell phone, tablet, T.V. size). Have them smell popcorn or hear angelic music. Kinesthetic feelers may want the place cold at 65, wrapped in a heavy blanket with a dog on their lap; auditory people may want background music; visual people may want dimmed lights, etc.

You will now watch yourself on the sofa/chair is the other you watches the movie

from picture 1 [symbol] all the way through to picture 2 [symbol] .

You as the observer will only watch the other you watch the movie. Do you understand?

[symbol]

10) You are now going to watch your movie of [symbol] in black and white, starting at [symbol] (Picture 1) and ending at [symbol] (Picture 2), taking you only 30 seconds; after 30 seconds, I will say, when you are done watching yourself. Are you ready? Go.

After 30 seconds, stop them if they are still watching them and check-in. How was that for you? How was that for Jesus?

11) What were you doing while the other you watched your movie? How was that for you on the sofa/chair watching the movie?

Make this observation pattern occur 3-5 times.

Words spoken to the Client are in bold.

Coach notes are in italics. These words are for the coach only, not told to the Client.

Do not move on until the Client is comfortable with this step. Repeat as needed. (At least three observations of the dissociated movie should occur, but as many as necessary.) Add adjustments if necessary, such as Shrinking the T.V. to the size of a cell phone, a stamp, and turning it off, making it more difficult to see and hear.

“Since it's your movie, tell yourself to pick up the remote, turn the picture, and sound off; the DVD will just run in the background.”

I will tell you during the 30-second movie to hold your breath for 10 seconds at some point; when I say breath, keep watching yourself over there.

Now, you will step into the end of your movie as yourself into

[symbol]

(picture 2) in black and white; you will re-associated in the scene reliving the experience. You will be seeing it through your own eyes and hearing it through your ears. Do you understand?

After they are associated, let them signal when they are ready to run the movie backward and associate from the last scene in picture two, black and white, to the first scene in picture one in color.

Listen to my instructions before doing anything. From inside the experience in a moment, you are going to move backward, from the end of the movie, in only two seconds, undoing all the actions of the event(s) to the beginning of the movie, where it ends in color, where you are safe before anything happens. You will stop at [symbol] or Picture 1 as it transitions back into color. As you start, I will say, “Whoooshhh,” and—just that fast—relive the entire movie backward. Do you understand?

[symbol]

Repeat instructions as needed. Do this a minimum of three times. 14) Please give me a thumbs up or nod when ready to move backward in time from the end of the movie to the beginning. (Pause) Here we go. Whoooshhh!

Draw out “whoooshhh” for two seconds. D.O. this 3-5 times before moving to 14.

15) Did you make it back to [symbol] or Picture 1? [symbol] Did Picture 1 transition back to color? [symbol] (Go back to line 14 for 3 repetitions minimum)

Repeat until the process is completed easily by the Client. Run this rewind at least 3-5 times, but as many as necessary.

Words spoken to the Client are in bold.

Coach notes are in italics. These words are for the coach only, not told to the Client.

16) Go ahead and think about the event that has you feeling excited and positive emotions and on a scale from 0 to 10, with 0 being no excited and positive emotions and 10 being extraordinarily strong excited and positive emotions relating to an elevation in status. Where are you now? [symbol] (If 0 leave it alone session is over, if more than 0 go to line 17 and continue)

If the Client's Subjective Excitement Score (SES) are three or below, they are ready for Phase Two. If the SES are at four (4) or above, return to Step 8 and repeat until the score is three or fewer.

If running multiple positive traumas, check the SES of each event. The same rules apply: three or less.

(3) or fewer reset in the move to phase 2, 4, or more, move back to the dissociated movie at line 7, and continue.

### Phase Two—Resetting The Emotion

Do you have a memory where you were humbled or remember watch someone perform a very humbling act? [symbol] Go back and relive the experience again.

Pause to give the Client time to truly feel the power of the moment).

Can you feel the humility now? [symbol] Feel the humility as long as you can.

Pause and ensure they can feel the emotions of the humility before moving on.

Think about the event that has been feeling excited or elevated in status. Where are you now on a scale from 0 to 10, with 0 being no excited or positive emotions and 10 being extraordinarily strong excited or positive emotions relating to an elevation in status

[symbol]

(If there is still a residual SES after line 18 take them back to line 17 and associate them into the feelings.)

Physically Anchor the feeling of humility in the heart area for 10-15 seconds.

Practice Visuals Exercise

Create a picture of yourself in school. Can you see that? Now drain the color and make it black and white. Is that done? Good, this is picture 1.

Create a memory of yourself grocery shopping after picture 1. That will be your movie called “grocery shopping.”

Create a picture of yourself at home after you are finished grocery shopping. Can you see that? Now drain the color and make it black and white. Is that done? Good, that is picture 2.

Can you see yourself sitting in your Livingroom on a sofa as if 3rd person?

Take picture 1 and put it on your TV, make sure it is black and white. Is it there?

You will observe yourself sitting on the sofa watching the movie of grocery shopping from picture 1 all the way to picture 2. The you on the sofa will watch the movie in 30 seconds. Ready, go. (3 repetitions)

Enter the last scene of the movie in picture 2 fully associated in black and white, seeing through your own eyes, hearing through your own ears and in a moment, you will go from the end of the movie in picture 2 all the way to the beginning of the movie in picture 1 where it will return to color in only 2 seconds. Does this make sense?

It will be like you're moving backwards undoing the actions of the event, walking backwards, running backwards, and being sucked through a wind tunnel ending at the beginning of the movie in color. It will make a sound like whooosh and I will make that sound pacing the 2 seconds. Are you ready? Go whooosh. Did you make it to the beginning in color? (repeat 5 times)

Associate into a better event where you can feel happiness, joy, peace, etc. As if you are reliving the experience. Can you feel the positive emotions? Good now imagine a dial in the lower corner turn it to the right and let it get 10 times more intense.

Study Group 1
614 Clients with PTSD
PTSD Symptom Scale Interview Version-5 (PSSI-5)

| | | | |
|---|---|---|---|
| Males: | 259 | | |
| Females: | 355 | | |
| Pre-scores: | 40.15 | | |
| Post scores: | 7.12 | Avg. sessions: | 1.27 |

| Study Group 2 | | Study Group 2 | |
|---|---|---|---|
| 322 Clients with PTSD | | Clients with Anxiety | |
| PTSD Checklist-5 (PCL-5) | | General Anxiety Disorder-7 (GAD7) | |
| Males: | 188 | Males: | 188 |
| Females: | 134 | Females: | 134 |
| pre-scores: | 45.93 | pre-scores: | 13.34 |
| post-scores: | 4.97 | post-scores: | 1.825 |
| Avg. sessions: | 1.24 | Avg. sessions: | 1.24 |

Study Group 3
92 Clients with Depression
Patient Health Questionaire-9 (PHQ9)

| | |
|---|---|
| Males: | 45 |
| Females: | 47 |
| Pre-scores: | 12.14 |
| Post scores: | 2.75 |
| Avg. sessions: | 1 |

The PEER RESCUE PROTOCOL
1. Create a picture of your birth in your mind with someone safe holding you. make it bright, colorful, and super focused, and feel their embrace, let me know when that is done. That image will stay there in color.
2. Move forward in your life from your birth to a traumatic moment/or a significant negative emotion of your life, let me know when you are there. Are you there? [symbol]
3. Drop into the memory, see what you saw, hear what you heard and feel what you felt at the time, as you are in the memory how intense do the emotions rise to on a0-10 scale with 0 being no negative emotions related to fear, terror, and helplessness (or significant negative emotion) and 10 the most intense? [symbol] . What do you want to call this memory (Significant Negative Emotion use order of the list and guide them to find the emotion of anger, rage, etc.) and how old are you? Name of event: Emotion: [symbol] Age: [symbol] SUDS: [symbol] BREAK STATE
4. Move far enough away from the traumatic memory or negative emotion so that you can barely make it out but not feel the emotions as you do this turn it black and white. Let me know when you are there and this is done. Are you there? [symbol] Good
5. Turn around away from the memory and as you do so you turn to see yourself as an observer and look directly into your own eyes. This moment is safe and is comforting to you.
6. What would you teach yourself about those moments, the learnings of which would allow you to let go quickly, easily, and effortlessly let the emotions related to event fall away, preserving the learnings, because they are useful, the emotions are not? [symbol] [symbol] [symbol] [symbol]
7. Listen to my instructions first. The observer self will now put their hands on your shoulders, smile and take you back inside your body and then all the way back to the color picture of you at your birth before anything bad happened. As you do this the emotions will fall away quickly, easily and effortlessly, Ready? [symbol] Here we go, wooooooooosh. Did you make it to birth in color? [symbol] Good.
8. Move forward from birth taking the observer self with you and with all the learnings given by your observer self, to just before the event, apply the new learnings of (Repeat line 6 learnings) so you can quickly and easily, and effortlessly let go of the emotions. After the memory look for any other memories after where you felt the same emotions, releasing them along the way and then come back to the present day when you are ready. OK, Go. Let me know when you are back to the present.

### Break State

9. Now go back to the traumatic memory/Significant Negative Emotion of: [symbol] , do you feel any emotions now or perhaps not? [symbol] If you do how intense are they on a 0-10 scale with 10 being the most intense? [symbol] (Over 3 go back to line 14 for 3 more sets, if emotions are still present go to line 4 after you distance them from the event so they can't feel the emotions and add new learnings for the specific memory and install new learnings. If SUDS is 0 then do line 10)
10. Think about the best moment of your life, a moment you can feel the positive emotions of love, peace, Joy, or other good feelings. let me know when you can feel them. [symbol] Good, now that you can feel them imagine a dial in the lower right corner, turn the dial to the right and let the feelings get 10 times more powerful, for vibrant, more focus and more color and sit there for a minute and enjoy them.
11. Drop back into the memory of [symbol] (Check each Trauma/Negative Emotions) and let me know if you can feel the emotions of fear, terror or helplessness or other negative emotions. On a 0-10 scale with 10 being the most intense where are you now? [symbol] (if O you are finished. Start over with the next trauma or negative emotion)

Create a picture of your birth in your mind with someone safe holding you, make it bright, colorful, and super focused, and feel their embrace, let me know when that is done. That image will stay there in color.

Move forward in your life from your birth to the first traumatic moment [(or to a Significant Negative Emotion of)] of your life, let me know when you are there. Are you there?

Drop into the memory, see what you saw, hear what you heard and feel what you felt at the time, as you are in the memory how intense do the emotions rise to on a 0-10 scale with 0 being no negative emotions related to fear, terror, and helplessness (Or Significant Negative Emotion of) and 10 the most intense as you feel it presently not then? What do you want to call this memory and how old were you? (Write down below then) BREAK STATE use line 4 by taking them back to birth until they no longer feel the emotions.

(Always return them to birth before line 4 moving to the next event)

(Then move them forward to future events using language in lines 2-3 with Break States between)

Break State

First event: Age: SUDS:

Break state.

Next event: Age: SUDS:

Break state.
4. Go back to the moment of your birth, are you there? Good now move forward to the next Traumatic memory (Or significant negative emotion of) (write above on list) (When all traumas or significant emotions are listed with names, age and SUDS go to line 5.)
5. Move far enough away from the last memory in the group of traumatic memories (or Significant Negative Emotions) so that you can barely make it out and not feel the emotions and turn the image black and white. Let me know when you are there. Are you there? Good

GET LEARNING NEXT
6. What would you teach yourself about those moments, the learnings of which would allow you to let go quickly, easily, and effortlessly all the emotions related to all the events and preserve the learnings, because they are beneficial, the emotions are not?
7. Listen to my instructions before beginning. You will reassociate back inside your body and then rewind all the way back to the color picture of you at your birth in 2 seconds before anything bad happened as you do this the emotions will fall away quickly, easily and effortlessly. Ready? Here we go, whooooooooosh. Did you make it to birth in color? Good.
8. Move forward from birth taking all the new learnings given by yourself, to just before each event, apply the new learnings of (Repeat line 6 learnings) so you can quickly and easily, and effortlessly let go of the emotions. (Guide them through each negative emotion memories/traumatic memories)

After the last memory look for any other memories after where you felt the same emotions, releasing them along the way and then come back to the present day when you are ready. BREAKSTATE
9. Now go back to the traumatic memory of, do you feel any emotions now or perhaps not? If you do how intense are they on a 0-10 scale with 10 being the most intense? (Write new SUDS for each memory on the list) (Over 3 go back to line 1gaining additional learnings present go to line after you distance them from the event so they cannot feel the emotions and add new learnings for the specific memory and install new learnings.)
10. Think about the best moment of your life, a moment you can feel the positive emotions of love, peace, Joy, or other good feelings. let me know when you can feel them. Good, now that you can feel them imagine a dial in the lower right corner, turn the dial to the right and let the feelings get 10 times more powerful, for vibrant, more focus and more color and sit there for a minute and enjoy them.
11. Drop back into the memory of (Check each Trauma/Emotions on the list) and let me know if you can feel the emotions of fear, terror or helplessness or other negative emotions. On a 0-10 scale with 10 being the most intense where are you now? (If 0 you can move back to line 4 and work on the next batch of emotions or traumas and check in on each moment and emotion, if they are 0 keep moving through them, if there is still a residual move them away from the moment and install new learnings until 0).

IF all 0s then this session is complete, you could do another batch of traumas/emotions or end the session. You can end with a grounding exercise and add breath.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A method for addressing posttraumatic stress and other negative emotional states in a client, the method comprising:
guiding the client through a Trauma Resiliency Protocol (TRP) and an Emotions Management Process (EMP) by a coach, wherein the TRP and EMP utilize a combination of visualization techniques, dissociation, neuro-linguistic programming (NLP), and reimprinting of emotions;
initiating the process by building rapport with the client and identifying a traumatic event or negative emotional state;
instructing the client to create a mental image of a safe moment before the traumatic event, and turning the image into black and white;
guiding the client to dissociate from the traumatic event by visualizing the event as a black and white movie, and observing themselves watching the movie from a third-person perspective;
leading the client to reimprint the traumatic memory by associating into a positive memory and amplifying the positive emotions;
conducting specific grounding exercises to ensure the client's emotional stability throughout the process;
using both secular and faith-based scripts to personalize the process based on the client's beliefs and preferences;
evaluating the client's distress levels using specific metrics including PSSI-5, PCL-5, PHQ-9, and GAD-7 before and after the TRP and EMP sessions;
repeating the steps of dissociation, reimprinting, and grounding until the client's distress levels are significantly reduced or eliminated; and
providing non-clinical interventions and peer coaching to make the method accessible and practical for a wide range of users, including military personnel, first responders, and individuals suffering from PTSD and other negative emotional states.
